(19) United States
(12) Patent Application Publication
(10) Pub. No.: US 2026/0207355 A1
(43) Pub. Date: Jul. 23, 2026

(54) PYRAMID COUPLER FOR USE WITH ARTIFICIAL LIMB

(71) Applicant: Bulldog Tools, Inc., Lewisburg, OH (US)

(72) Inventor: Robert Meyer, Lewisburg, OH (US)

(21) Appl. No.: 19/456,811

(22) Filed: Jan. 22, 2026

Related U.S. Application Data
(60) Provisional application No. 63/748,095, filed on Jan. 22, 2025.

(51) Int. Cl. A61F 2/78

(52) U.S. Cl. CPC A61F 2/78

(57) ABSTRACT

According to aspects of the present disclosure, a pyramid coupler for use with an artificial limb comprises a base and a coupler. The base includes a support portion, wherein the support portion includes four apertures, each aperture located on a protrusion of the support portion, and a domed portion that does not include a ledge and is disposed in a center of the base. The coupler is coupled to the domed portion at a center of the domed portion and includes a post, a flattened portion of the post, and an aperture. The post flares out as it extends away from the base, and the flattened portion ends before reaching a top of the coupler. Further, the aperture is at the top of the coupler that extends along an axis of the coupler.

### CROSS REFERENCE TO RELATED APPLICATIONS

This application claims the benefit of U.S. Provisional Patent Application Ser. No. 63/748,095, filed Jan. 22, 2025, having the title PYRAMID COUPLER FOR USE WITH ARTIFICIAL LIMB, the entire disclosure of which is hereby incorporated herein by reference.

### BACKGROUND

Various aspects of the present disclosure relate generally to prosthetic implants and more specifically to a pyramid coupler for a prosthetic implant.

A prosthetic implant is an artificial device that replaces a missing body part, including limbs. Most limb prosthetic implants include a pyramid coupler that couples the artificial limb to the user.

### BRIEF SUMMARY

According to aspects of the present disclosure, to aspects of the present disclosure, a pyramid coupler for use with an artificial limb comprises a base and a coupler. The base includes a support portion, wherein the support portion includes four apertures, each aperture located on a protrusion of the support portion, and a domed portion that does not include a ledge and is disposed in a center of the base. The coupler is coupled to the domed portion at a center of the domed portion and includes a post, a flattened portion of the post, and an aperture. The post flares out as it extends away from the base, and the flattened portion ends before reaching a top of the coupler. Further, the aperture is at the top of the coupler that extends along an axis of the coupler.

According to further aspects of the present disclosure, in some embodiments, the top of the coupler connects to the post of the coupler via a radius.

Also, aspects of the present disclosure, in several embodiments, the top of the coupler is circular.

Moreover, further aspects of the present disclosure relate to including a shelf between protrusions of the base. In some embodiments, the pyramid coupler includes four protrusions but only three shelves, leaving a space between two of the protrusions empty. In various embodiments, the shelves are concave, convex, straight, or combinations thereof.

Yet further aspects of the present disclosure include numerous embodiments of the pyramid coupler where the base includes a chamfered aperture for connecting a retention component between the coupler and the base. Several embodiments lack an o-ring coupler between the coupler and the base.

### BRIEF DESCRIPTION OF THE SEVERAL VIEWS OF THE DRAWINGS

FIG. 1 is a side view of a diagram of a pyramid coupler, according to various aspects of the present disclosure;

FIG. 2 is a top view of a diagram of the pyramid coupler of FIG. 1, according to various aspects of the present disclosure;

FIG. 3 is an angled bottom view of a diagram of the pyramid coupler of FIGS. 1-2, according to various aspects of the present disclosure;

FIG. 4 illustrates an embodiment of the pyramid coupler where a support portion is arched and includes four legs with apertures, according to various aspects of the present disclosure;

FIG. 5 illustrates an embodiment of the pyramid coupler where a support portion is arched and includes four legs (longer than in FIG. 4) with two apertures per leg, according to various aspects of the present disclosure;

FIG. 6 illustrates an embodiment of the pyramid coupler where a support portion is arched and includes four legs (longer than in FIG. 4) with multiple apertures per leg, according to various aspects of the present disclosure;

FIG. 7 illustrates an embodiment of the pyramid coupler where a support portion is arched and includes four legs with apertures (similar to FIG. 4) and includes a threaded portion internal to the coupler, according to various aspects of the present disclosure;

FIG. 8 illustrates an embodiment of the pyramid coupler where a support portion is a flat circle and includes apertures that are wider than apertures of other embodiments, according to various aspects of the present disclosure;

FIG. 9 illustrates an embodiment of the pyramid coupler where a support portion includes a hub where the apertures extend in a radial direction, which allows coupling to an artificial limb, according to various aspects of the present disclosure;

FIG. 10 illustrates an embodiment of the pyramid coupler where a support portion includes a hub (similar to the coupler in FIG. 9, except longer) where the apertures extend in a radial direction, which allows coupling to an artificial limb, according to various aspects of the present disclosure;

FIG. 11 illustrates an embodiment of the pyramid coupler where a support portion is a cuff that can be cinched over an artificial limb and secured with a fastener through an aperture, according to various aspects of the present disclosure;

FIG. 12 illustrates an embodiment of the pyramid coupler where a support portion is similar to the support portion of FIG. 2 and includes the apertures and a radial aperture, according to various aspects of the present disclosure;

FIG. 13 illustrates an embodiment of the pyramid coupler where a support portion is a top portion of a sphere and includes apertures, according to various aspects of the present disclosure;

FIG. 14 illustrates an embodiment of the pyramid coupler where a domed portion is offset in a length direction on a circular support portion that includes protrusions, ledges, and apertures, according to various aspects of the present disclosure;

FIG. 15 illustrates an embodiment of the pyramid coupler where a domed portion is offset in a length direction on an ovoid support portion that includes protrusions, ledges, and apertures, according to various aspects of the present disclosure;

FIG. 16 illustrates an embodiment of the pyramid coupler where a domed portion is offset in a length direction on a rectangular support portion that includes protrusions, ledges, and apertures, according to various aspects of the present disclosure;

FIG. 17 illustrates an embodiment of the pyramid coupler where a domed portion is offset in a diagonal direction on a rectangular support portion that includes protrusions, ledges, and apertures, according to various aspects of the present disclosure;

FIG. 18 illustrates an embodiment of the pyramid coupler where a domed portion is offset in a length direction on an ovoid support portion that includes apertures with sloped portions, according to various aspects of the present disclosure;

FIG. 19 illustrates an embodiment of the pyramid coupler where a domed portion is offset in a length direction on a rectangular support portion such that the domed portion protrudes past the support portion. The support portion includes protrusions and apertures, according to various aspects of the present disclosure;

FIG. 20 illustrates an embodiment of the pyramid coupler where a domed portion is offset in a length direction on an angled support portion that includes apertures with sloped portions and a cross-shaped void, according to various aspects of the present disclosure;

FIG. 21 illustrates an embodiment of the pyramid coupler where a domed portion is offset in a length direction on an angled support portion that includes wide apertures and a cross-shaped void, according to various aspects of the present disclosure;

FIG. 22 illustrates an embodiment of the pyramid coupler where a domed portion is in a recessed portion that is offset in a length direction on an angled support portion that includes wide apertures and a cross-shaped void, according to various aspects of the present disclosure;

FIG. 23 illustrates an embodiment of the pyramid coupler where a domed portion is offset in a length direction on a rectangular support portion that includes protrusions and apertures, according to various aspects of the present disclosure; and

FIG. 24 illustrates an embodiment of the pyramid coupler where a domed portion is offset in a length direction on a flat, rectangular support portion that includes apertures, according to various aspects of the present disclosure.

### DETAILED DESCRIPTION

A pyramid coupler is used to couple an artificial limb to a user. Most pyramid couplers include a base and a coupler, where the coupler tapers out as it extends from the base. That coupler then couples to a socket on the user. According to aspects herein, several improvements to a pyramid coupler are disclosed.

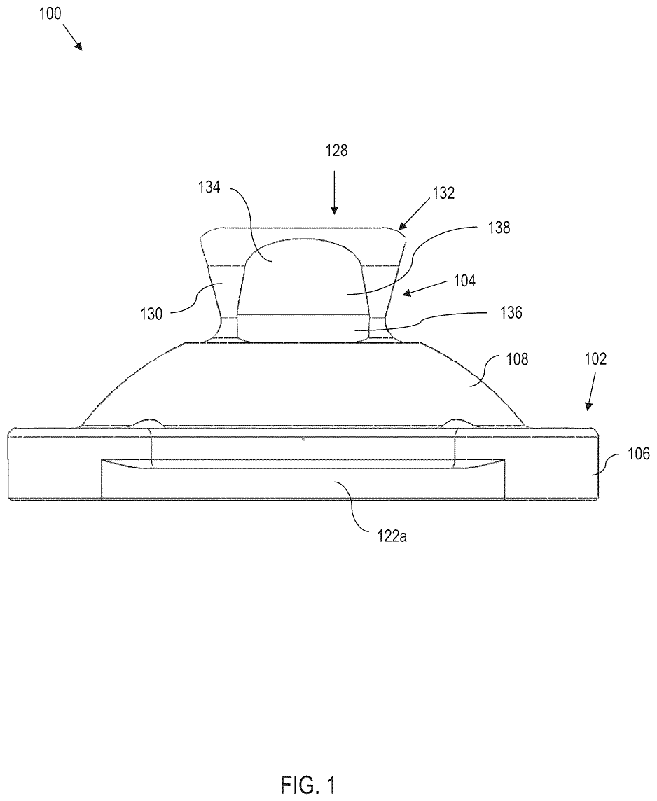

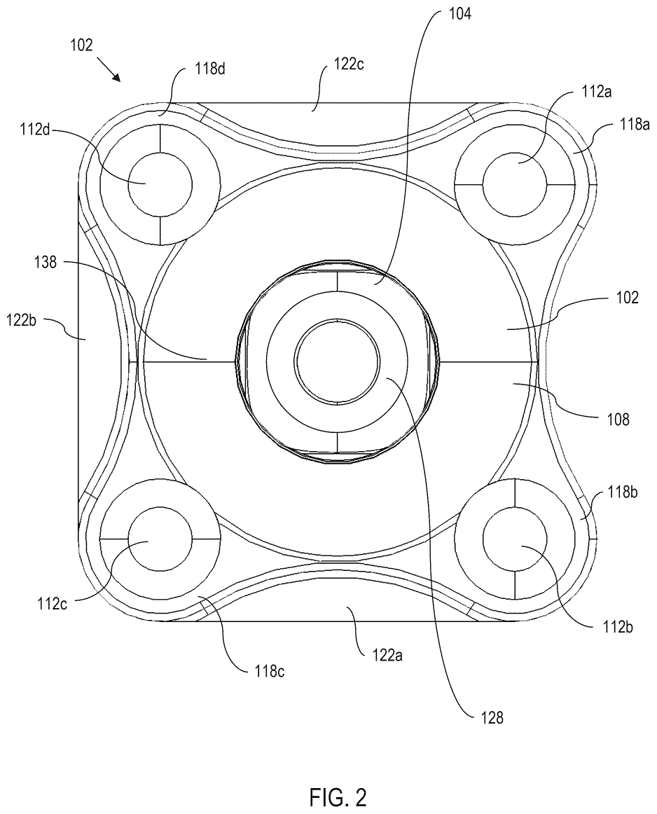

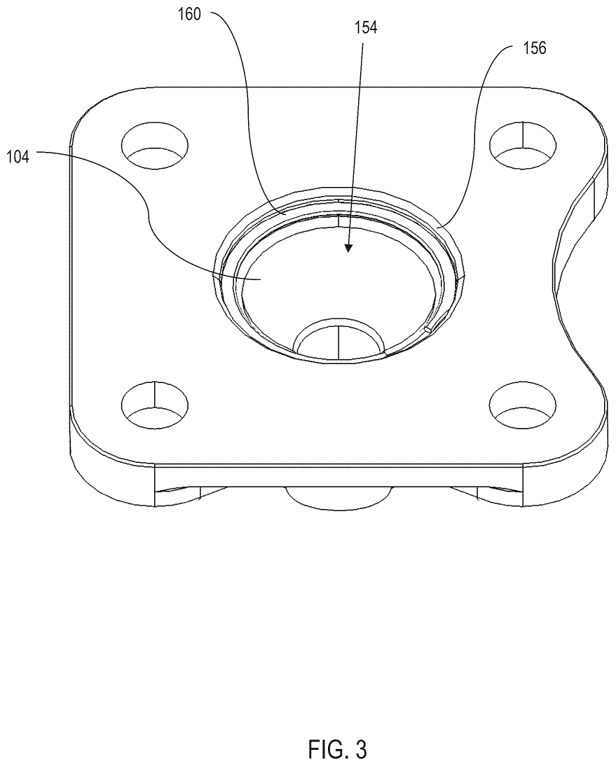

Turning now to the figures, and in particular FIGS. 1-2, a top view of a pyramid coupler 100 is shown. The pyramid coupler 100 includes a base 102 and a coupler 104. The base 102 includes a support portion 106 and a domed portion 108. The support portion 106 includes apertures 112a-d (collectively 112) that are used to couple the pyramid coupler to an artificial limb (e.g., via a torsion adapter). While four apertures 112a-d are shown in FIG. 2, any number of apertures 112 may be present in other embodiments of the pyramid coupler. The domed portion 108 is in a center of the base 102, but the domed portion 102 may be skewed from the center in some embodiments. Further, the domed portion 108 includes an aperture (see 154, FIG. 3) in which the coupler 104 resides, as shown below in reference to FIG. 3. Moreover, the domed portion 108 continues radially from the support portion to the aperture in which the coupler 104 resides. In other words, there is no ledge (or flattened portion) of the dome before the coupler 104, as there is on existing pyramid couplers. This domed portion 108 with no ledge strengthens the connection between the coupler and the domed portion.

With continued reference to FIGS. 1-2, many embodiments of the pyramid coupler 100 include protrusions 118a-d (collectively 118), which surround the apertures 112 on the support portion 106. In FIG. 2, while the number of protrusions 118 is equal to the number of apertures 112, such an implementation is not required. For example, one of the protrusions 118 may not include an aperture 112. Moreover, in many embodiments there is a shelf 122a-c (collectively 122) between two of the protrusions 112. As shown in FIG. 2, in a preferred embodiment, there are three shelves 122a-c with one space between two protrusions devoid of a shelf. However, there may be as many shelves as spaces between protrusion or less than the number of spaces between protrusions (e.g., 2, 4, 5, etc.). The shelves 122 may be linear (as shown in FIG. 2), convex (e.g., to make the pyramid coupler more of a circle shape instead of the square shape of FIG. 2), concave, or combinations thereof. These shelves 122 provide strength to the pyramid coupler while a missing shelf (e.g., three shelves for four protrusions) allows some compliance.

The coupler 104 includes a flattened top 128 the couples to a post 130 via a radius 132. However, in other embodiments, the flattened top 128 couples directly to the post 130 or indirectly to the post 130 via a chamfer. Further, the post 130 includes a flattened portion 134 that extends from the domed portion 108 of the base 102. The flattened portion 134 can be one plane that flares out or (as shown in FIG. 1) may include a lower portion 136 that tapers in and an upper portion 138 that flares out. In either configuration, the flattened portion 134 ends before reaching the flattened top 128, which makes the flattened top circular when looking down on it (see FIG. 2). The coupler further includes an aperture 140 that extends along an axis of the coupler 104.

As discussed above the coupler 104 couples to the domed portion 108 of the base 102 via the aperture in the domed portion 108. Turning to FIG. 3, a bottom 150 of the base 102 is shown in an isometric view. The bottom 150 includes the apertures 112a-d discussed above. Further, the bottom 150 includes the aperture 154 that includes a chamfer 156 to allow the coupler 104 to slide into the aperture 154 easier. The coupler is then secured via a retention component 160 (e.g., a retaining ring) without a need for an o-ring. This is an advantage, because the o-ring may provide too much friction when locking the pyramid coupler, and reduces surface area of a contact between the coupler and the domed portion, which is undesirable. Therefore, the pyramid couplers described herein without an o-ring provide advantages over existing solutions.

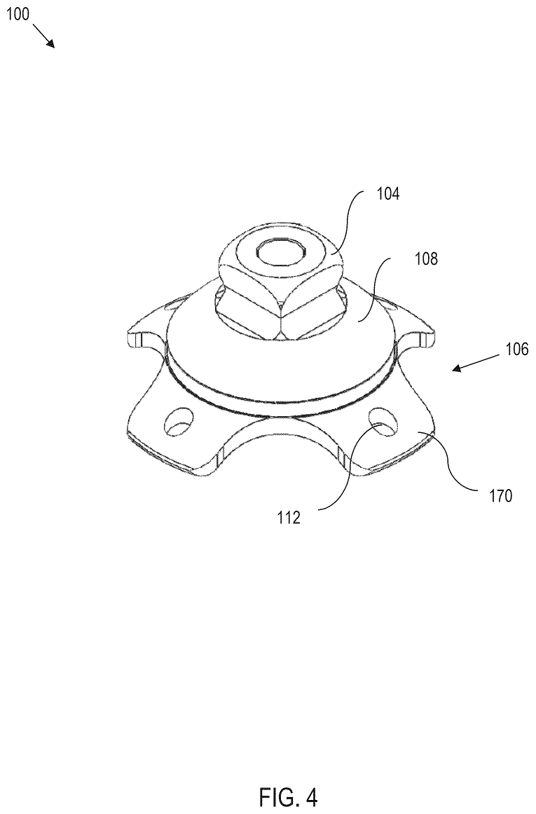

While the support portion 106 is shown with four apertures and generally square-shaped, other support portions 106 may be used as shown in FIGS. 4-24 that include embodiments of the pyramid coupler 100. For example, in FIG. 4, the support portion 106 is arched and includes four legs 170 with apertures 112. The coupler 104 couples to the domed portion 108 as described above.

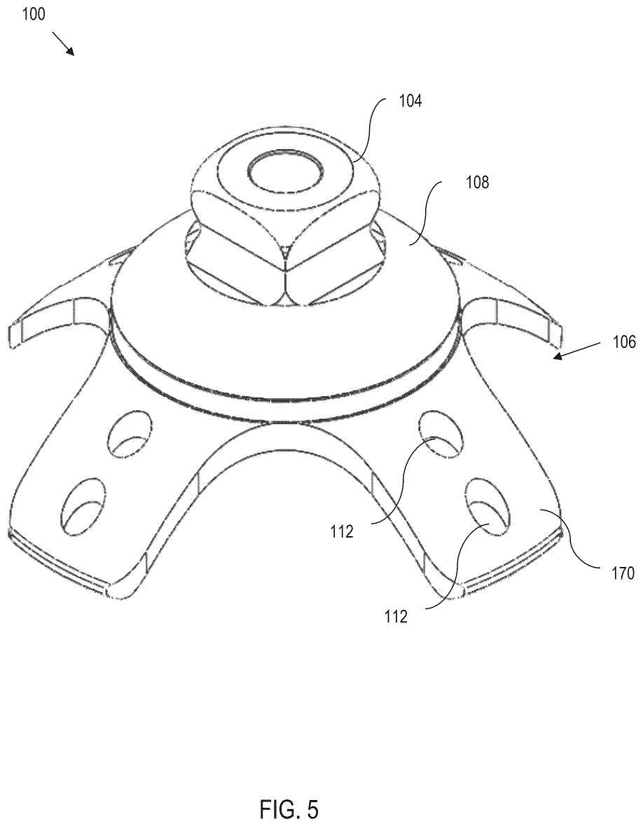

In FIG. 5, the support portion 106 is arched and includes four legs 170 (longer than in FIG. 4) with two apertures 112 per leg. The coupler 104 couples to the domed portion 108 as described above.

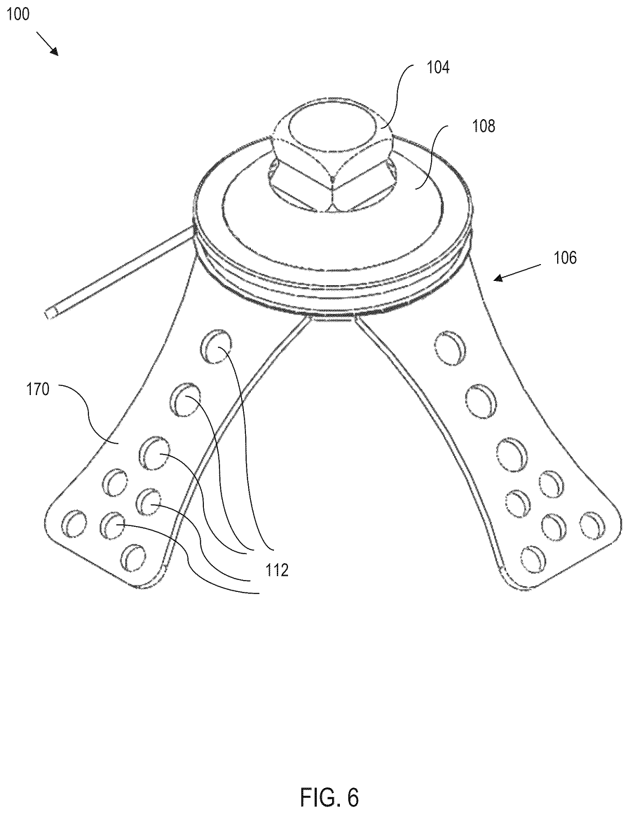

In FIG. 6, the support portion is arched and includes four legs 170 (longer than in FIG. 4) with multiple apertures 112 per leg. The coupler 104 couples to the domed portion 108 as described above.

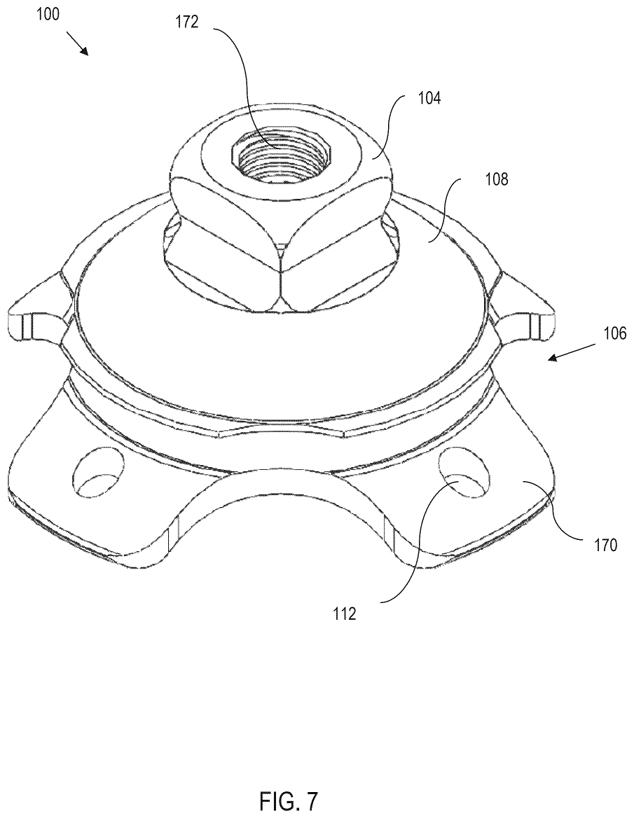

In FIG. 7, the support portion 106 is arched and includes four legs 170 with apertures 112 (similar to FIG. 4) and includes a threaded portion internal 172 to the coupler 104. The coupler 104 couples to the domed portion 108 as described above.

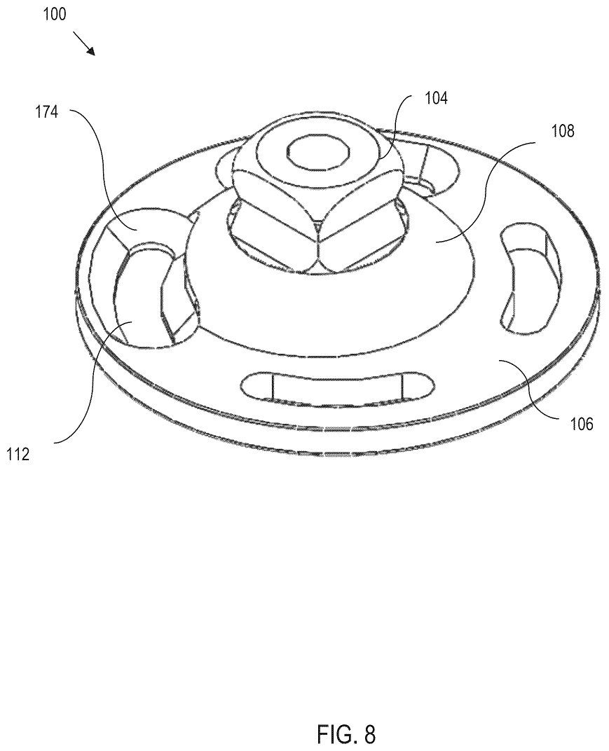

In FIG. 8, the support portion 106 is a flat circle and includes apertures 112 that are wider than other apertures. Further, some of the apertures 112 include a sloped portion 174 aid in fastening the pyramid coupler 100 to an artificial limb, as discussed above. The coupler 104 couples to the domed portion 108 as described above.

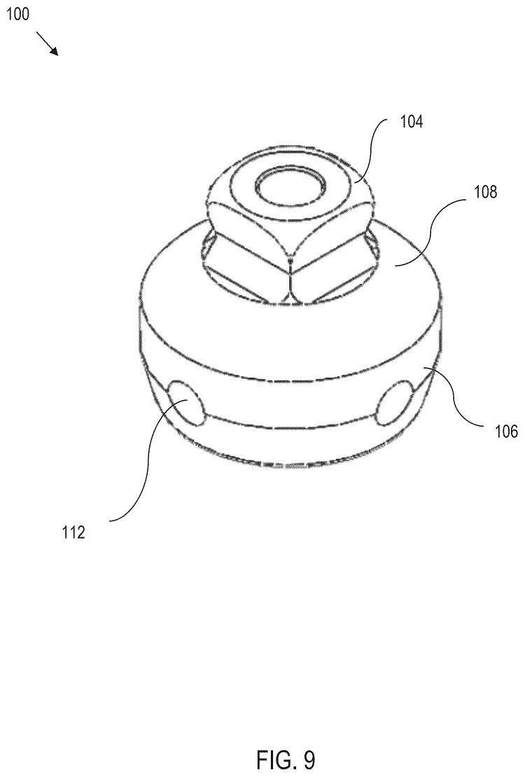

In FIG. 9, the support portion 106 includes a hub where the apertures 112 extend in a radial direction, which allows coupling to an artificial limb. The coupler 104 couples to the domed portion 108 as described above.

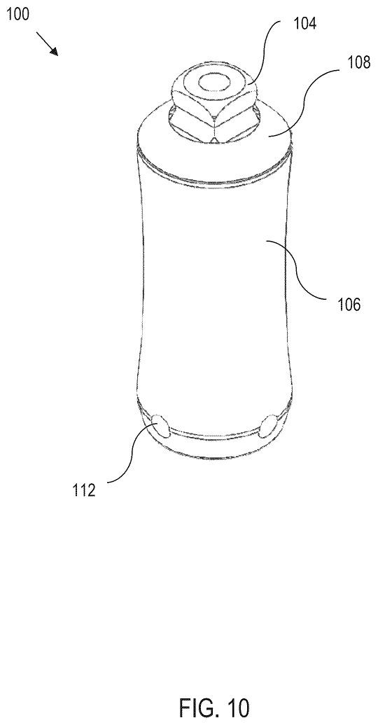

In FIG. 10, the support portion 106 includes a hub (similar to the coupler in FIG. 9, except longer) where the apertures 112 extend in a radial direction, which allows coupling to an artificial limb. The coupler 104 couples to the domed portion 108 as described above.

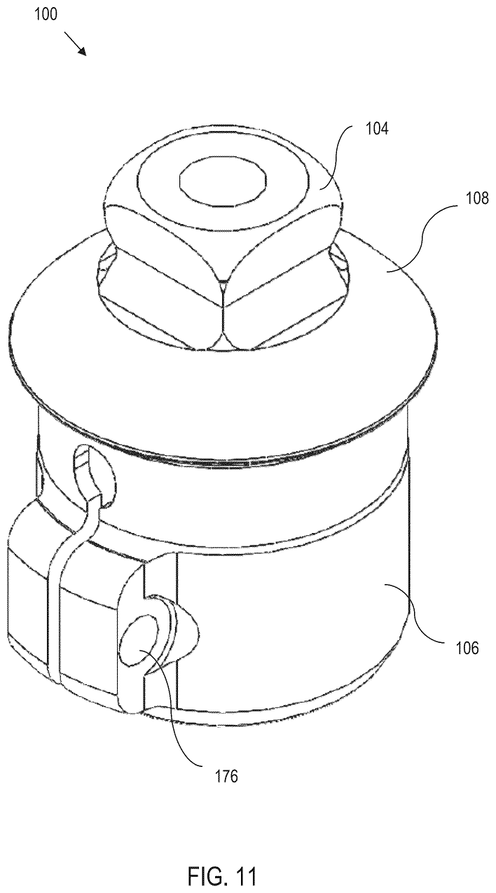

In FIG. 11, the support portion 106 is a cuff that can be cinched over an artificial limb and secured with a fastener through an aperture 112. The coupler 104 couples to the domed portion 108 as described above.

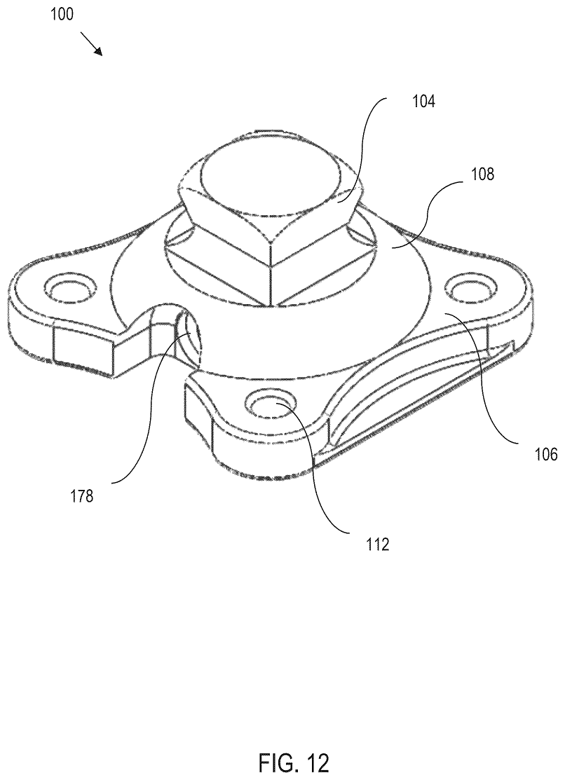

In FIG. 12, the support portion 106 is similar to the support portion of FIG. 2 and includes the apertures 112 and a radial aperture 176. The coupler 104 couples to the domed portion 108 as described above.

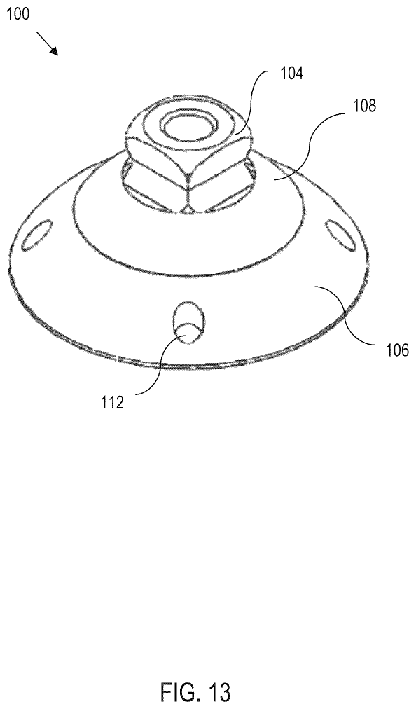

In FIG. 13, the support portion 106 is a top portion of a sphere and includes apertures 112. The coupler 104 couples to the domed portion 108 as described above.

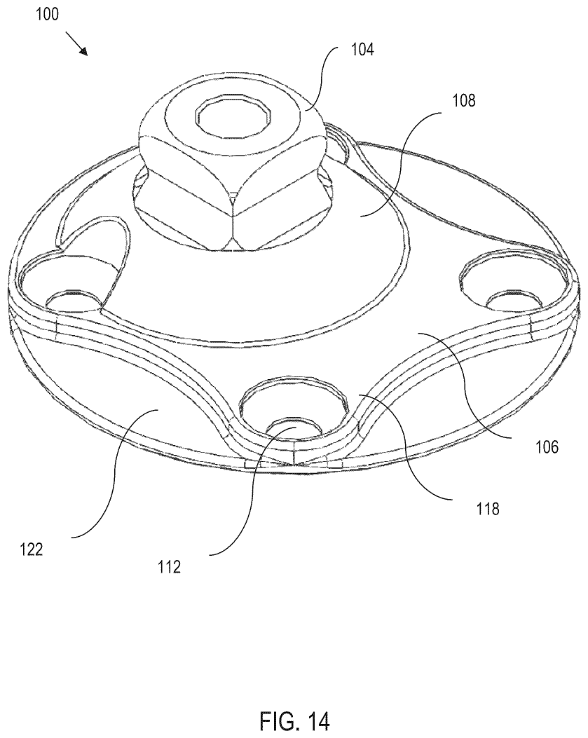

While, in FIGS. 1-13, the domed portion 108 is in a center of the support portion 106, as discussed above, the domed portion 108 may be located off-center (i.e., skewed from center) as well. For example, in FIG. 14, the domed portion 108 is offset in a length direction on a circular support portion 106 that includes protrusions 118, ledges 122, and apertures 112. The coupler 104 couples to the domed portion 108 as described above.

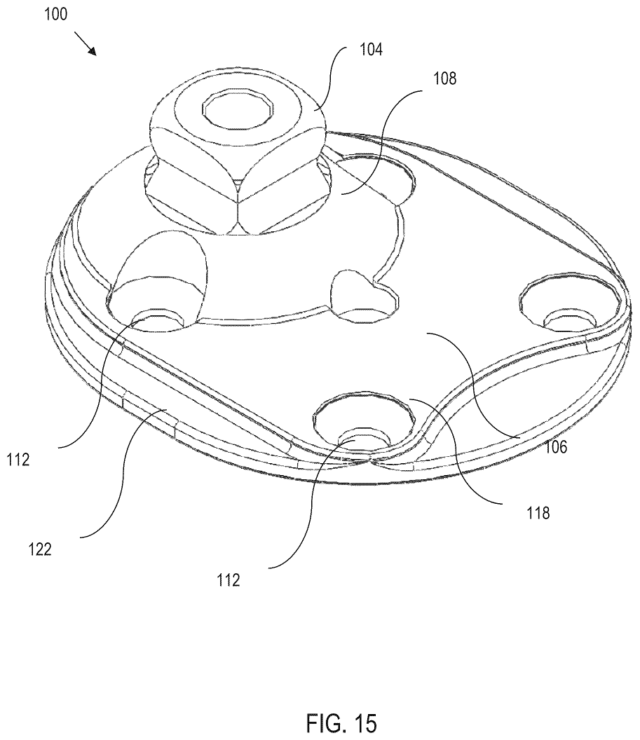

In FIG. 15, the domed portion 108 is offset in a length direction on an ovoid support portion 106 that includes protrusions 118, ledges 122, and apertures 112. The coupler 104 couples to the domed portion 108 as described above.

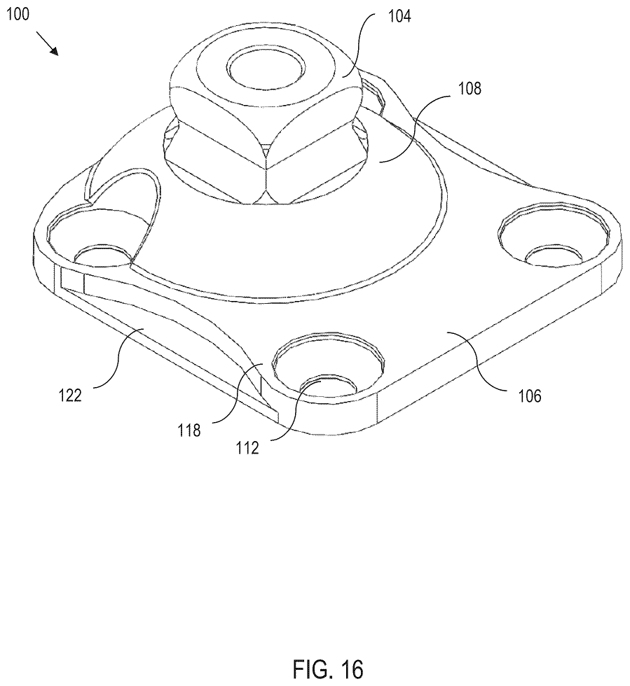

In FIG. 16, the domed portion 108 is offset in a length direction on a rectangular support portion 106 that includes protrusions 118, ledges 122, and apertures 112. The coupler 104 couples to the domed portion 108 as described above.

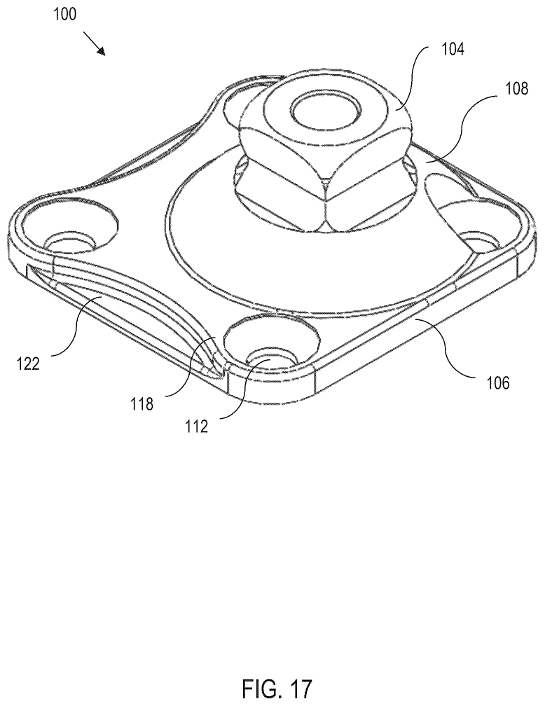

In FIG. 17, the domed portion 108 is offset in a diagonal direction on a rectangular support portion 106 that includes protrusions 118, ledges 122, and apertures 112. The coupler 104 couples to the domed portion 108 as described above.

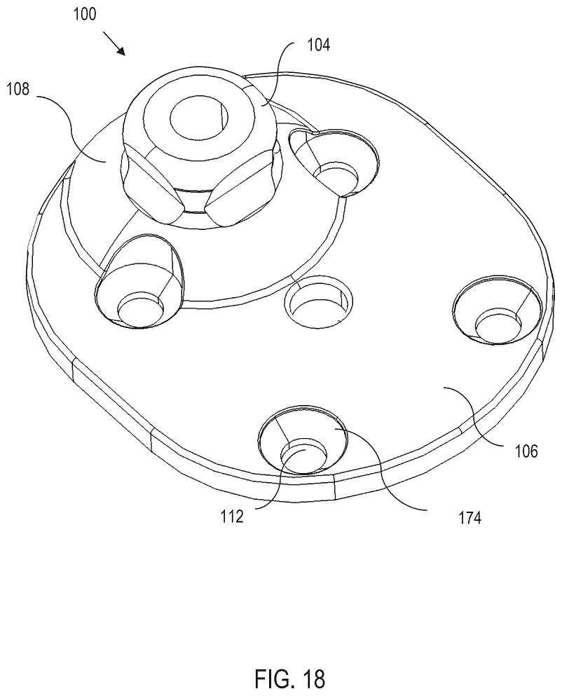

In FIG. 18, the domed portion 108 is offset in a length direction on an ovoid support portion 106 that includes apertures 112 with sloped portions 174. The coupler 104 couples to the domed portion 108 as described above.

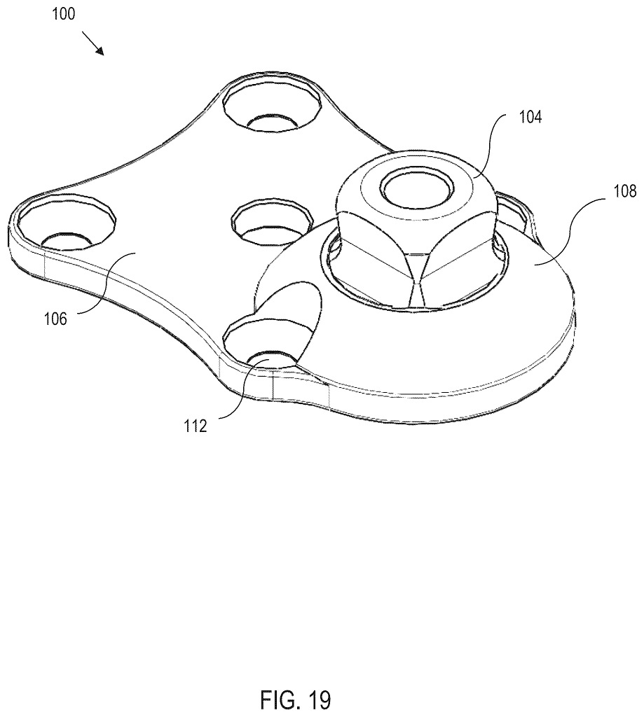

In FIG. 19, the domed portion 108 is offset in a length direction on a rectangular support portion 106 such that the domed portion protrudes past the support portion. The support portion includes protrusions 118 and apertures 112. The coupler 104 couples to the domed portion 108 as described above.

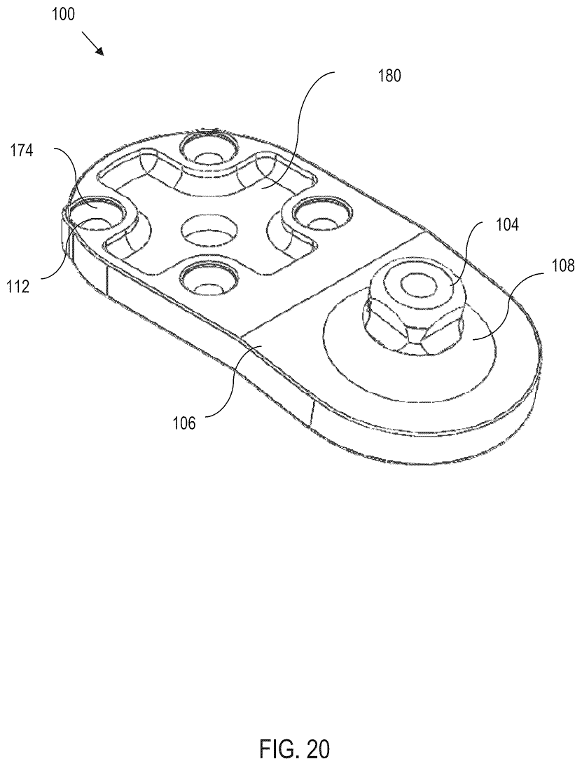

In FIG. 20, the domed portion 108 is offset in a length direction on an angled support portion 106 that includes apertures 112 with sloped portions 174 and a cross-shaped void 180. The coupler 104 couples to the domed portion 108 as described above.

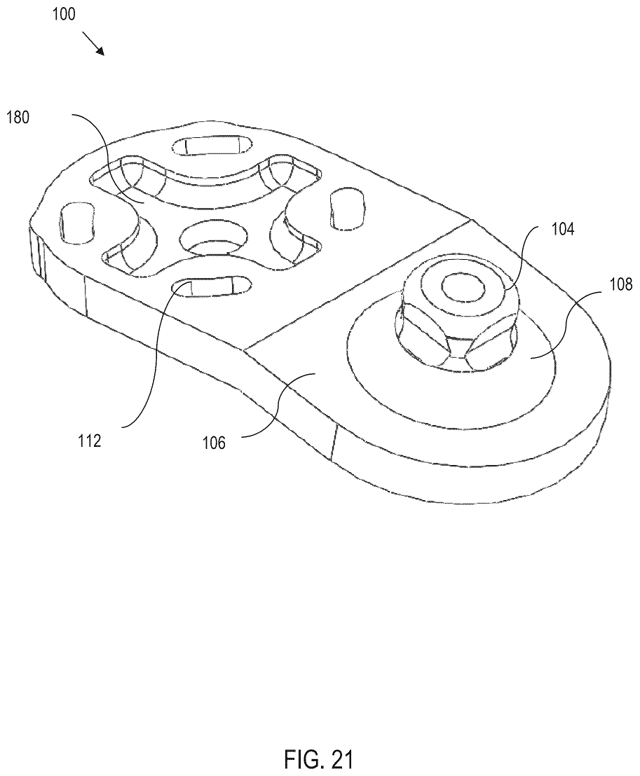

In FIG. 21, the domed portion 108 is offset in a length direction on an angled support portion 106 that includes wide apertures 112 and a cross-shaped void 180. The coupler 104 couples to the domed portion 108 as described above.

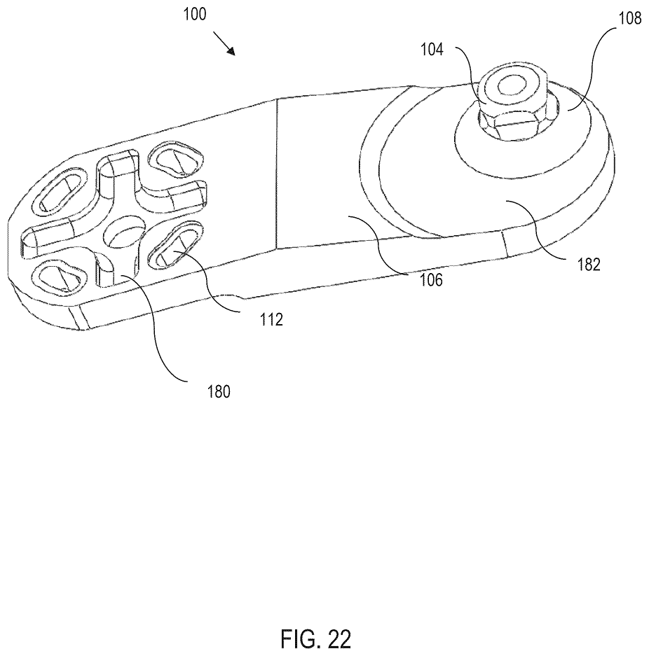

In FIG. 22, the domed portion 108 is in a recessed portion 182 that is offset in a length direction on an angled support portion 106 that includes wide apertures 112 and a cross-shaped void 180. The coupler 104 couples to the domed portion 108 as described above.

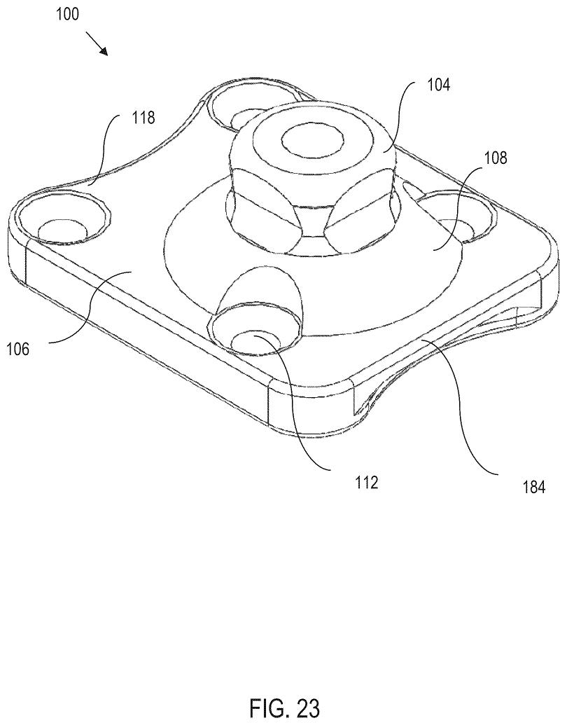

In FIG. 23, the domed portion 108 is offset in a length direction on a rectangular support portion 106 that includes protrusions 118, apertures 112, and an inverted shelf 184. The coupler 104 couples to the domed portion 108 as described above.

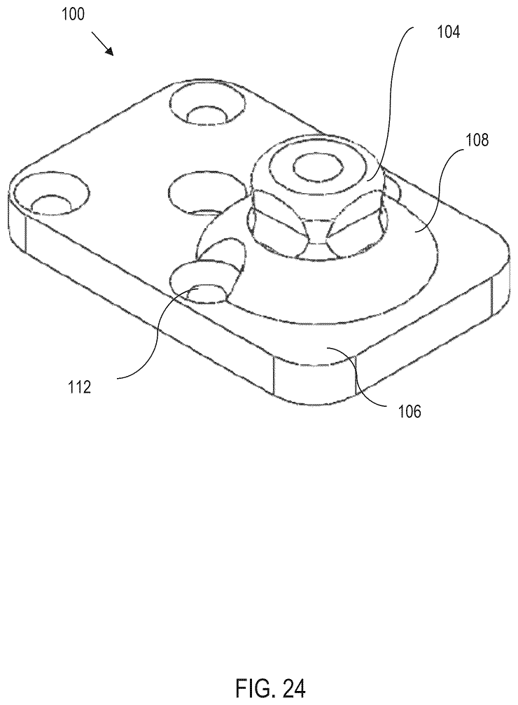

In FIG. 24, the domed portion 108 is offset in a length direction on a flat, rectangular support portion 106 that includes apertures 112. The coupler 104 couples to the domed portion 108 as described above.

Regardless of the shape of the support portion 106 and whether the domed portion 108 is centered or off-center, the coupler portion 104 couples to the domed portion 108 in a similar way, with the chamfer to allow the coupler 104 to slide into the aperture 154 and secured with a retention component without the need for an o-ring.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the disclosure in the form disclosed. Many modifications and variations will be apparent to those of ordinary skill in the art without departing from the scope and spirit of the disclosure. Aspects of the disclosure were chosen and described in order to best explain the principles of the disclosure and the practical application, and to enable others of ordinary skill in the art to understand the disclosure for various embodiments with various modifications as are suited to the particular use contemplated.

What is claimed is:

1. A pyramid coupler for use with an artificial limb, the pyramid coupler comprising:
a base including:
a support portion; and
a domed portion that does not include a ledge, wherein the domed portion is disposed in a center of the base; and
a coupler coupled to a center of the domed portion, wherein the coupler includes:
a post that flares out from the domed portion;
a flattened portion on the post that extends from a base of the coupler but ends before a top of the coupler; and
includes an aperture at the top of the coupler that extends along an axis of the coupler;
wherein the coupler is not integral with the base.

2. The pyramid coupler of claim 1, wherein the top of the coupler connects to the post of the coupler via a radius.

3. The pyramid coupler of claim 1, wherein the top of the coupler is circular.

4. The pyramid coupler of claim 1, further including a shelf between two protrusions of the base.

5. The pyramid coupler of claim 4, further including four shelves, wherein each shelf is between two of the protrusions of the base.

6. The pyramid coupler of claim 4, wherein the shelf is convex.

7. The pyramid coupler of claim 4, wherein the shelf is concave.

8. The pyramid coupler of claim 4, wherein the shelf is straight.

9. The pyramid coupler of claim 4, further including only three shelves, wherein each shelf is between two of the protrusions of the base.

10. The pyramid coupler of claim 1, wherein the coupler tapers out from the domed portion starting at the domed portion.

11. The pyramid coupler of claim 1, wherein the coupler tapers out from the domed portion starting at a point above the domed portion.

12. The pyramid coupler of claim 1, where there is no o-ring between the coupler and the base.

13. The pyramid coupler of claim 1, wherein the base includes a chamfered aperture for connecting a retention component between the coupler and the base.

14. The pyramid coupler of claim 1, wherein the domed portion is centered on the support portion.

15. The pyramid coupler of claim 1, wherein the domed portion is off-center on the support portion.

16. A pyramid coupler for use with an artificial limb, the pyramid coupler comprising:
a base including:
a support portion; and
a domed portion that does not include a ledge, wherein the domed portion is disposed in a center of the base; and
a coupler coupled to a center of the domed portion, wherein the coupler includes:
a post that flares out from the domed portion; and
a flattened portion on the post that extends from a base of the coupler but ends before a top of the coupler.
